(19) United States
(12) Patent Application Publication
(10) Pub. No.: US 2008/0119752 A1
(43) Pub. Date: May 22, 2008

(54) CALORIMETRY

(75) Inventor: Michael Flanagan, Manchester (GB)

Correspondence Address: HUSCH BLACKWELL SANDERS LLP, 720 OLIVE STREET, SUITE 2400, ST. LOUIS, MO, 63101, US

(73) Assignee: NUTREN TECHNOLOGY LIMITED, Lancashire (GB)

(21) Appl. No.: 11/681,163

(22) Filed: Mar. 1, 2007

(30) Foreign Application Priority Data
Nov. 22, 2006 (GB) 0623245.8

(51) Int. Cl. A61B 5/083

(52) U.S. Cl. 600/531

(57) ABSTRACT

According to the present invention in a first aspect, there is provided an apparatus for determining the type of fuel burnt by a user, the apparatus comprising an oxygen sensor and a carbon dioxide sensor, and wherein the oxygen sensor and the carbon dioxide sensor are operable to establish the type of fuel burnt by a user of the apparatus.

### CROSS-REFERENCE TO RELATED APPLICATION

This application claims the benefit of United Kingdom Application No. 0623245.8, filed on Nov. 22, 2006 in the United Kingdom Intellectual Property Office, the disclosure of which is incorporated herein by reference.

### FIELD OF THE INVENTION

The present invention relates to apparatus for calorimetry and methods for processing calorimetric data.

### BACKGROUND TO THE INVENTION

In the various physiological processes undertaken by a user (human, animal, etc.) different fuels are burnt. These fuels can be broadly categorised into fat, protein and carbohydrates. In the user's body such fuels are reacted with oxygen to produce energy with the by-products of carbon dioxide and water. In an aerobic state the body will convert fuel as indicated by the following exemplary equations:

C₆H₁₂O₆+6O₂→6 H₂O+6CO₂−2872KJ (carbohydrate reaction) Eq1.

C₆H₃₂O₂(Palmitic Acid)+23O₂→16H₂O+16CO₂+9795KJ (fat reaction) Eq2.

Indirect calorimetric techniques involve measuring the chemical by-products of various physiological processes to study the energy produced during metabolism in humans and animals. The techniques can be used, for example, by sport or nutritional scientists for the diagnosis of metabolic disorders and for calculating nutritional requirements. However, such calorimeters are only used to provide an indication of the metabolic rate of a user and provide no indication of any other characteristics of the user's metabolism such as to determine the type of fuel being burnt.

It is an aim of the preferred embodiments of the present invention to provide an apparatus that complements present calorimeters and provides an indication of different characteristics associated with a user's metabolism.

### SUMMARY OF THE INVENTION

According to the present invention in a first aspect, there is provided an apparatus for determining the type of fuel burnt by a user, the apparatus comprising an oxygen sensor and a carbon dioxide sensor, and wherein the oxygen sensor and the carbon dioxide sensor are operable to establish the type of fuel burnt by a user of the apparatus.

The oxygen sensor may be any suitable sensor. Suitable oxygen sensors include a zirconia, an electrochemical or Galvanic, an infrared, an ultrasonic or a laser sensor.

The carbon dioxide sensor nay be any suitable sensor. Suitable sensors include infrared sensors and chemical gas sensors.

Suitably, the apparatus further comprises a volume sensor operable to establish the type of fuel burnt by a user.

The volume sensor may be any suitable sensor. Suitable sensors include flow sensors and pressure sensors.

Suitably, the apparatus further comprises a processing means configured to establish the type of fuel burnt by a user from at least cue measurement from the oxygen sensor and at least one measurement from the carbon dioxide sensor. Suitably, the processing means is further configured to establish the type of fuel burnt by a user from at least one measurement from the volume sensor.

Suitably, the processing means is configured to determine the fuel burnt as a ratio of the carbon dioxide sensor measurement to the oxygen sensor measurement. Suitably, the ratio is a ratio of the amount of carbon dioxide produced by a user to the amount of oxygen consumed by a user. Alternatively, the ratio is a ratio of the volume of carbon dioxide produced by a user to the volume of oxygen consumed by a user. Suitably, the processing means is configured to compare the ratio to predetermined respiratory quotient values to determine the type of fuel burnt by a user. Suitably, the processing means is configured to match the ratio to a predetermined respiratory quotient value to establish the type of fuel burnt by a user. Suitably, the apparatus further comprises a display configured to display type of fuel burnt by a user.

Suitably, the processing means is configured to establish the amount of oxygen consumed by a user from the amount of oxygen inhaled and exhaled by a user. Alternatively, the processing means is configured to establish the volume of oxygen consumed by a user from the volume of oxygen inhaled and exhaled by a user.

Suitably, the processing means is configured to establish the amount of carbon dioxide produced by a user from the amount of carbon dioxide inhaled and exhaled by a user. Alternatively, the processing means is configured to establish the volume of carbon dioxide produced by a user from the volume of oxygen inhaled and exhaled by a user.

Suitably, the processing means is configured to determine the oxygen consumed by a user by subtracting the inhaled breath measurement from the exhaled breath measurement. Suitably, the processing means is configured to determine the carbon dioxide produced by a user by subtracting the exhaled breath measurement from the inhaled breath measurement.

Suitably, the processing means is configured to use a predetermined inhaled breath measurement to establish the oxygen consumed and carbon dioxide produced by a user. Suitably, the apparatus comprises a timer configured to determine an inhaled breath time. Suitably, the processing means is configured to determine the amount or volume of oxygen in an inhaled breath by multiplying the inhaled breath time by the appropriate predetermined inhaled breath measurement.

The predetermined inhaled breath measurement may correspond to the quantity of oxygen and carbon dioxide present in the air surrounding the device. The predetermined value can correspond to calibrated quantities or to quantities present in or around the device in use.

Suitably, the sensors are operable periodically. Alternatively, the sensors are operable continuously.

Suitably, the apparatus further comprises a breath direction sensor configured to establish if a user is inhaling or exhaling into the apparatus. Suitably, the processing means is configured to use the breath direction sensor readings to classify the oxygen sensor and carbon dioxide sensor readings into exhaled or inhaled breath measurements. Suitably, the processing means is configured to use the breath direction sensor readings to classify the volume sensor readings into exhaled or inhaled breath measurements.

Suitably, the apparatus is configured to determine the type of fuel burnt by a user from at least one breath. Suitably, the apparatus is configured to determine the type of fuel burnt by a user from a plurality of breaths. Suitably, the apparatus further comprises a timer, the timer configured to determine the amount time that a user is in fluid communication with the apparatus. Suitably, the apparatus further comprises a warning means configured to warn the user when, the have been in fluid communication with the device for a predetermined time.

Suitably, the apparatus is configured to determine the amount or volume of carbon dioxide produced joy averaging the carbon dioxide produced from each of the plurality of breaths. Alternatively, the amount or volume of carbon dioxide produced is determined by averaging the total amount or volume of carbon dioxide produced by a user with the total time that a user is in fluid communication with the apparatus. In another alternative, the total amount or volume of carbon dioxide produced is determined by averaging the total amount or volume of carbon dioxide produced with the total number of breaths a user delivers to the apparatus.

Suitably, the apparatus further comprises a housing and a first and a second fluid inlet. Suitably, the first fluid inlet is arranged in fluid communication with the oxygen sensor and/or the carbon dioxide sensor. Suitably, the second fluid inlet is arranged in fluid communication with the oxygen sensor and/or the carbon dioxide sensor. Suitably, the first fluid and/or the second fluid inlet is arranged in fluid communication with the breath direction sensor.

Suitably, the oxygen sensor and/or the carbon dioxide sensor are arranged between the first fluid inlet and the second fluid inlet. Suitably, the breath direction sensor is arranged between the first and the second fluid inlets.

Suitably, the first fluid inlet comprises the second fluid inlet. Suitably, the first fluid inlet is a tube. Suitably, the tube is rigid. Alternatively, the tube is flexible.

Suitably, the second fluid inlet is provided as at least one aperture on the tube.

Suitably, the apparatus further comprises a housing. Suitably, the housing comprises the first fluid inlet. Suitably, the housing comprises the second fluid inlet. Suitably, the first and second fluid inlets are arranged in fluid communication with the oxygen sensor and/or the carbon dioxide sensor. Suitably, the first and second fluid inlets are arranged in fluid communication with the breath direction sensor.

According to the present invention in a second aspect, there is provided a method of calculating the type of fuel burnt by a user, the method comprising the steps of:
(a) providing a apparatus comprising an oxygen sensor and a carbon dioxide sensor;
b) establishing the proportion of oxygen consumed by the user;
(c) establishing the proportion of carbon dioxide produced by a user; and
(d) establishing the type of fuel burnt by a user.

Suitably, in step (a) the apparatus provided is the apparatus according to the first aspect and the proportions established in steps (b) and (c) are an amount of oxygen consumed and carbon dioxide produced by a user.

### BRIEF DESCRIPTION OF THE DRAWINGS

For a better understanding of the invention, and to show how embodiments of the same may be carried into effect, reference will now be made, by way of example, to the accompanying diagrammatic drawings in which:

FIG. 1 shows a schematic side view of a first embodiment of the apparatus of the present invention;

FIG. 2 shows a schematic side view of a second embodiment of the apparatus of the present invention;

FIG. 3 shows a schematic side view of a third embodiment of the apparatus of the present invention;

FIG. 4 shows a schematic side view of a fourth embodiment of the apparatus of the present invention;

FIG. 5 shows a schematic side view of a fifth embodiment of the apparatus of the present invention;

FIG. 6 shows a schematic side view of a sixth embodiment of the apparatus of the present invention;

FIG. 7 shows a schematic side view of a seventh embodiment of the apparatus of the present invention; and

FIG. 8 shows a schematic side view of a eight embodiment of the apparatus of the present invention.

### DETAILED DESCRIPTION OF THE PREFERRED EMBODIMENTS

FIGS. 1 through 8 show the preferred embodiments of the present invention. The preferred embodiments each provide an apparatus 1-8 for determining the type of fuel burnt by a user. Each apparatus 1-8 is complementary to the use of indirect calorimeters and provides additional information other than a user's metabolic rats that can, for example, be used by sports or nutrition scientists, etc.

As indicated by Eq1. and Eq2. different amounts of oxygen (O₂) and carbon dioxide (CO₂) are consumed and produced during the various physiological processes undertaken within a user's body. The ratio of the different amounts of CO₂ and O₂ is known as the respiratory quotient (RQ), which can be calculated using the following equation:

$$RQ = \frac{{V}_{{CO}_{2}}}{{V}_{{O}_{2}}} = \frac{{M}_{{CO}_{2}}}{{M}_{{O}_{2}}} . \qquad Eq3$$

where Vco₂ is the volume of CO₂ produced per unit time and Mco₂ is the number of moles of CO₂ consumed per unit time; and

Vo₂ is the volume of O₂ produced per unit time and Mo₂ is the number of moles of O₂ consumed per unit time.

It has been found that particular RQ values correspond to the different types of fuel burnt by a user's body. Table 1 shows an example of different RQs for the different types of fuel:

TABLE 1

| FUEL | IRATORY QUOTIENT (RQ) |
|---|---|
| Fat | 0.71 |
| Protein | 0.8 |
| Carbohydrates | 1.0 |

Each of the apparatus 1-8 are operable to establish the type or types of fuel burnt by a user from the volume or number of moles of both oxygen and carbon dioxide present in a user's breath. An indication of the type of fuel burnt provides information complementary to information relating to the amount of energy consumed by a user.

FIG. 1 shows a first embodiment 1 of the apparatus of the present invention. The apparatus 1 comprises an oxygen (O₂) sensor 10, a carbon dioxide (CO₂) sensor 11, a processing means 12, a housing 13, a first fluid inlet 14 for receiving a user's exhaled breath, a second fluid inlet 15 for transmitting a user's inhaled breath to the user and a breath direction sensor 16. The apparatus 1 is operable to determine the type of fuel burnt by a user from the measurements of the O₂ sensor 10 and the CO₂ sensor 11.

The O₂ sensor 10 is used to establish the amount or moles of oxygen consumed by a user's body. The O₂ sensor 10 can be any suitable sensor, which includes a zirconia, an electrochemical or Galvanic, an infrared, an ultrasonic or a laser sensor.

The CO₂ sensor 11 is used to establish the amount or moles of CO₂ produced by a user's body. The CO₂ sensor 11 can be any suitable sensor, which includes infrared sensors, and chemical gas sensors.

The sensors 10, 11 are operable to measure a user's breath periodically. For example, the sensors 10, 11 are operable to measure and transmit a user's breath measurement in 1 ms intervals. The processing means upon receipt of each signal determines the amount of oxygen or carbon dioxide present in a user's breath upon each transmission and then upon completion of a user's breath the processing means determines the total oxygen and carbon dioxide present in a user's breath. The total content of the breath can be determined by multiplying the value associated with each transmission by 2 to compensate for the part of the breath not transmitted by the periodically operable sensor and then summing the inhaled breath measurements to produce a total inhaled breath measurement and summing the exhaled breath measurements to produce a total exhaled breath measurement.

The processing means 12 can also determine an inhaled breath time, i.e., the time taken for a user to inhale a breath, and an exhaled breath time, i.e., the time taken for a user to exhale a breath, from the number of sensor transmissions corresponding to the inhaled breath or the exhaled breath.

Alternatively, the sensors 10, 11 are continuously operable to measure a user's breath. When the sensors 10, 11 are continuously operable, the apparatus 1 can further comprise a timer to determine an inhaled breath time and an exhaled breath time. The continuously operable sensor provides inhaled breath measurements and exhaled breath measurements that correspond to total breath measurements.

It is of course possible to use any number of oxygen sensors 10 and/or carbon dioxide sensors 11.

The breath direction sensor 16 is employed by the apparatus 1 to aid establishing whether the breath is being inhaled or exhaled by the user. The processing means 12 uses the breath direction sensor readings to classify the oxygen sensor and carbon dioxide sensor readings as exhaled or inhaled breath measurements.

The breath direction sensor can be a flow sensor, or a pressure sensor, etc. The breath direction sensor 15 is positioned in the first fluid inlet 14.

The breath direction sensor 16 could, for example, also be provided using the oxygen sensor 10 and the carbon dioxide sensor 11 in conjunction with the processing means 12. In this example, the oxygen sensor 10 and carbon dioxide sensor 11 are positioned such that when a breath is exhaled or inhaled, the processing means 12 is configured to detect a detection sequence for the breath, i.e., when a breath is exhaled into the apparatus 1 the oxygen sensor 10 detects the breath before the carbon dioxide sensor 11 and the processing means 12 determines this detection sequence to be an exhaled breath, and when a breath is inhaled through the apparatus 1 the oxygen sensor 10 detects the breath after the carbon dioxide sensor 11 and the processing means 12 determines this detection sequence to be an inhaled breath. The processing means 12 then establishes the type of fuel burnt by a user using the inhaled/exhaled breath determinations. The processing means 12 determines the inhaled detection sequence and the exhaled detection sequence to be a breach sequence. Alternatively, the processing means 12 determines an exhaled detection sequence to be a breath sequence.

Alternatively, the carbon dioxide sensor and the oxygen sensor are positioned conversely to that described above and the processing means is adapted accordingly.

The sensors 10, 11 are operable to transmit the amount of O₂ or CO₂ in a user's inhaled breath and exhaled breath to the processing means 12. The processing means 12 then establishes the amount of oxygen consumed and the amount of carton dioxide produced by a user.

Alternatively, the sensors 10, 11 are operable to only transmit the amount of O₂ or CO₂ in a user's exhaled breath to the processing means 12. The processing means 12 then establishes the amount of oxygen consumed and the amount of carbon dioxide produced by a user using a user's exhaled breath measurements and predetermined inhaled breath measurements. The predetermined inhaled breath measurements correspond to the amount of oxygen and carbon dioxide present in the air surrounding the device and can be determined when the device is switched on.

The predetermined inhaled breath can also be factory calibrated and based on, for example, the quantities of oxygen and carbon dioxide present in air in standard conditions.

When the processing means establishes the amount of oxygen or carbon dioxide present using the predetermined inhaled breath measurement, the processing means determines the inhaled breath time. The processing means then multiplies the inhaled breath time by the predetermined breath measurement to determine the amount of oxygen and/or carbon dioxide present in a user's inhaled breath.

The processing means 12 can also establish the amount of oxygen or carbon dioxide present per unit time in a user's breath by determining the total oxygen and carbon dioxide present in a user's exhaled breath and inhaled breath dividing the inhaled breath measurements by the inhaled breath time and the exhaled breath measurements by the exhaled breath time.

Subsequently, the processing means determines the amount of oxygen consumed and carbon dioxide produced by a user. The amount of oxygen consumed is established by subtracting the amount of oxygen in an inhaled breath from the amount of oxygen measured in an exhaled breath. The amount of CO₂ produced is established by subtracting the amount of CO₂ measured in an exhaled breath from the amount of CO₂ in an inhaled breath.

The processing means 12 calculates a user's respiratory quotient as a ratio of the amount of carbon dioxide produced to the amount of oxygen consumed. The processing means 12 then compares the ratio with predetermined RQ values, such as those shown in Table 1, and matches the ratio to the nearest RQ value to establish the type of fuel burnt by a user, but not the amount of energy used by a user.

The apparatus 1 is operable in a single breath configuration and a multi-breath configuration.

In the single breath configuration, a single breath sequence, which is an inhaled breath and a subsequent exhaled breath, is used to determine the amount of oxygen consumed and the amount of carbon dioxide produced by user. The processing means 12 provides a user with a fast measurement from the signals of the oxygen sensor 10 and carbon dioxide sensor 11. The processing means 12 calculates the ratio from the single breath sequence to determine the type of fuel burnt by a user.

In the multi-breath configuration, the apparatus 1 is configured to establish the fuel burnt by a user from a plurality of breath sequences. In this alternative, the processing means 12 averages the signals from the oxygen sensor 10 and carbon dioxide sensor 11 to determine the fuel burnt by a user.

In the multi-breath configuration, there are a number of alternatives of determining the fuel burnt by a user. The processing means 12 can determine the fuel burnt by a user by averaging the total value of the oxygen and carbon dioxide sensor 10, 11 readings against a period of time that a user is in fluid communication with the device, or a total number of breath sequences delivered to the apparatus 1.

Alternatively, the processing means 12 determines the amount of oxygen consumed and the amount carbon dioxide produced in a single breath sequence, and then averages these amounts against the total number of breaths delivered by the user to the apparatus 1 to determine the fuel burnt by a user.

In a further alternative, the user apparatus 1 is used to determine the type of fuel burnt by a user at different periods of a day. For example, the apparatus 1 could be used to establish the type of fuel burnt by a user before and after eating, or in the morning and in the afternoon, etc. The results of the determination could be used to provide a sport or nutritional scientist with information suitable for determining a users dietary requirements.

The O₂ sensor 10, CO₂ sensor 11, processing means 12 and breath direction sensor 16 are provided in the housing 13.

The first and second fluid inlets 14, 15 are arranged in fluid communication with the oxygen sensor 10 and the carbon dioxide sensor 11, and the breath detection sensor 16. The housing 13 comprises the first and second fluid inlet 14, 15. The first and second fluid inlets 14, 15 are connected to the housing 13. The first and second fluid inlets 14, 15 can be formed integrally with or separately from the housing 13.

There are a number of ways in which the oxygen sensor 10, carbon dioxide sensor 11 and breath direction sensor 16 can be arranged. On such way is to arrange the oxygen sensor 10, carbon dioxide sensor 11 and breath direction sensor 16 between the first and second fluid inlets 14, 15. In this way, inhaled air is transmitted from the second fluid inlet 15 via the sensors to the user and exhaled air is transmitted from the user via the sensors to the second fluid inlet 15.

The first fluid inlet 14 shown in FIG. 1 is a tube. The tube is rigid. The second fluid inlet 15 can be provided as at least one aperture on the tube.

The tube can of course be flexible, for example, a flexible hose. The flexible hose can be any suitable length, for example, the hose can between 5 cm to 100 cm in length.

The apparatus 1 also comprises a mouthpiece (not shown). The mouthpiece is connected to the first fluid inlet 14 at the end of the first fluid inlet 14 opposed to the sensors 10, 11. The mouthpiece is formed integrally with the first fluid inlet 14. Alternatively, the mouth piece can be separable from the first fluid inlet 14 to, for example, enable the mouthpiece to be changed for different users. The mouthpiece can also be a mask.

The apparatus 1 can also be calibrated for measurement purposes. There are many ways in which the apparatus 1 can be calibrated including setting the apparatus to establish total amount of oxygen or carbon dioxide present in a user's breath in whole numbers. These values can then be used by the processing means to calculate the fuel burnt by a user.

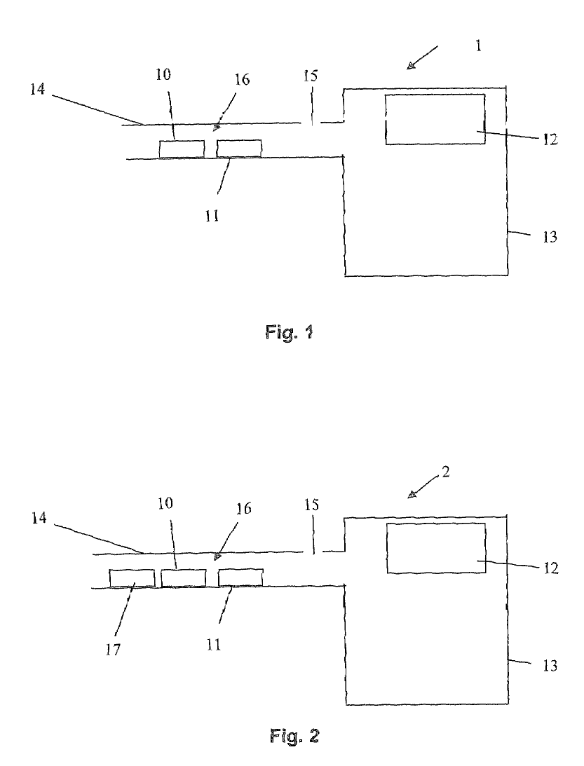

FIG. 2 shows the second embodiment 2 of the apparatus. The apparatus 2 further comprises the features of the first aspect and a volume sensor 17 that measures the volume of an exhaled breath or an inhaled and an exhaled breath. The volume sensor 17 can be any suitable device capable of producing a measurement suitable for the calculation of a volume of a breath. The volume sensor 17 measures the total volume of a user's breath.

The volume sensor 17 can be a flow sensor. The flow sensor measures the amount of flow through the fluid inlet per unit time. The flow measurement can, for example, be used to calculate the velocity of a breath. The velocity along with the known area of the first fluid inlet 14 is then used to determine the volume of a breath.

Alternatively, the volume sensor 17 can be a pressure sensor. The pressure sensor measures the pressure exerted by a user's breath. The pressure measurement is then used to calculate the volume of a breath using, for example, the ideal gas equations.

The processing means 12 is configured to establish the total volume of user's exhaled or inhaled breath from the measurement of the volume sensor 17. In the second embodiment 2, the oxygen sensor 10 and the carbon dioxide sensor 11 are operable to measure the proportion or percentage of oxygen and carbon dioxide present in a user's exhaled and inhaled breath. Then, using the readings from the oxygen sensor 10 and carbon dioxide sensor 11, the total volume of oxygen and carbon dioxide present in a user's inhaled or exhaled breath can be established by multiplying the percentage of oxygen or carbon dioxide measured with the volume sensor measurements for a user's exhaled or inhaled breath.

Alternatively, the oxygen sensor 10 and carbon dioxide 11 are operable to only transmit the measurements of a user's exhaled breath to the processing means 12. The processing means 12 then establishes the volume of oxygen consumed and the volume of carbon dioxide produced by a user by multiplying the user's exhaled breath measurements and the predetermined inhaled breath measurements for oxygen and carbon dioxide with the user's inhaled and exhaled breath volume sensor measurements.

The predetermined inhaled breath measurements can be determined as described for the first embodiment.

Subsequently, the volumes are used to establish the type or types of fuel burnt by a user as a ratio of the volume of carbon dioxide consumed to the volume of oxygen consumed. The processing means 12 establishes the type of fuel burnt by a user by comparing the ratio with predetermined respiratory quotient values, such as those shown in Table 1, and matches the ratio to the nearest predetermined respiratory quotient value.

The breath direction sensor 16, as described for the first embodiment, can be used by the processing means to determine whether the user's breath is an inhaled breath or an exhaled breath.

The apparatus 2 is operable in a single breath configuration and a multi-breath configuration. Apart from being configured to determine the volume of oxygen consumed and the volume of carbon dioxide produced by user, the processing means 12 is operable to determine the fuel burnt by a user as described for the first embodiment.

The apparatus 2 is operable to establish the type of fuel burnt by a user from the readings output by oxygen sensor 10 and the carbon dioxide sensor 11. As the output from the sensors will generally be an electrical signal, the apparatus 1 can be calibrated. There are many ways in which the apparatus can be calibrated including setting the oxygen and carbon dioxide readings on scale of 1 to 100. Using such as scale, the scaled values can, for example, indicate the proportion of a user's inhaled/exhaled breath that is oxygen and/or carbon dioxide. The scaled values can then be used by the processing means to calculate the volume of oxygen consumed and volume of carbon dioxide produced to establish the fuel burnt by a user.

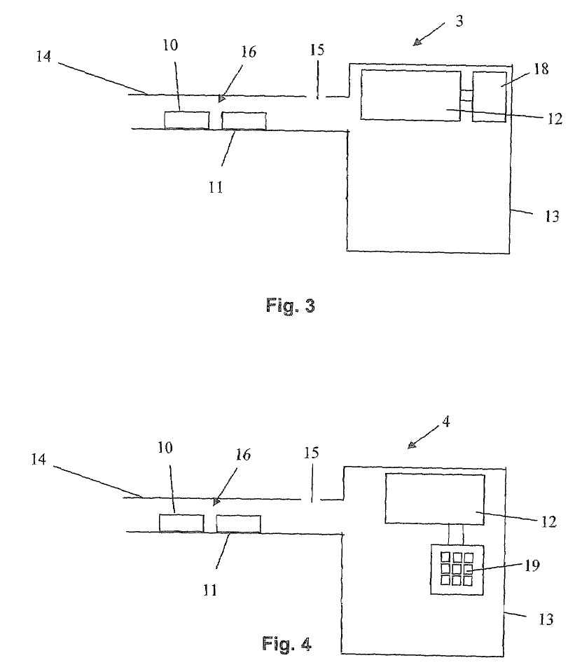

FIG. 3 shows a third embodiment of the apparatus 3. The apparatus 3 comprises the features of the first embodiment and a storage means 18 to store the signals from the oxygen sensor 10 and carbon dioxide sensor 11. The processing means 12 uses the stored values to calculate the fuel used by a user.

The third embodiment 3 can further comprise the features of the second embodiment 2.

The storage means 18 can be used to store the measurements from the sensor 10, 11 in such a way that they are accessible after use of the apparatus 3. For example, if the processing means 12 is provided in an external apparatus such as a computer, the processing means 12 can access the stored oxygen sensor 10 measurements when the apparatus 3 is connected to the computer.

The apparatus 3 is used by a single user. However, the apparatus 3 can be used by multiple users, and the storage means 18 can be configured to retrievably store multiple individual user configurations, i.e., a user's parameters, previous fuel burning data, etc.

FIG. 4 shows a fourth embodiment 4 of the apparatus. The apparatus 4 comprises the features of the first embodiment and an input means 19. The input means 13 is provided, for example, for a user to enter user parameters such as their height, weight, age and gender. The constant CC is also influenced by these factors, just as metabolism in general. The user parameters are used by the processing means 12 to more accurately determine the type of fuel burnt by a user.

The fourth embodiment 4 can further comprises the features of the second embodiment 2.

The input means 19 is provided on the housing 12. Alternatively, the input means 19 can be provided on an external apparatus such as a computer. The input means 19 can be, for example, a key pad suitable for the user to input user parameters.

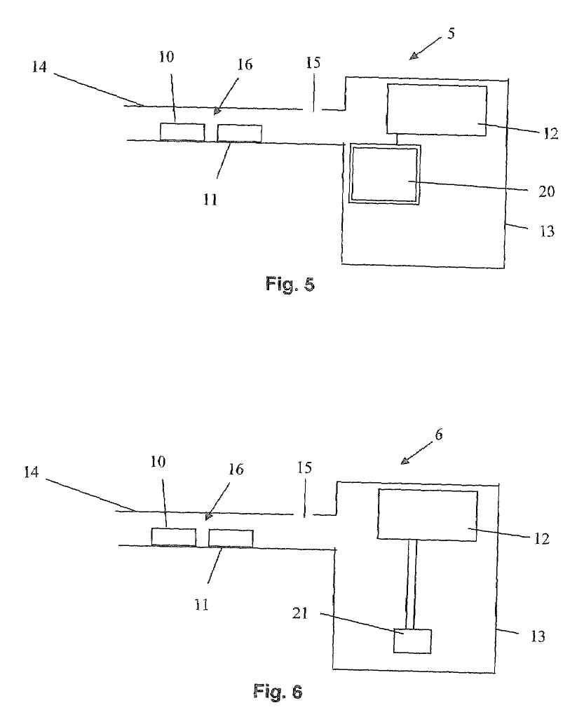

FIG. 5 shows a fifth embodiment 5 of the apparatus. The apparatus 5 comprises the features of the first embodiment and a display 20. The display 20 displays, for example, a user parameter and the type of fuel burnt by a user.

The fifth embodiment 5 can further comprise the features of the second embodiment 2.

The display 20 is a liquid crystal display provided on the housing. However, the display could be any suitable display means. For example, the display 20 could be provided using a personal computer. In another example, the display 20 could be a printer.

FIG. 6 shows a sixth embodiment 6 of the apparatus. The apparatus 5 comprises the features of the first embodiment and a timer 21. The timer 21 is provided to time the amount of time a user has been in fluid communication with the apparatus 5.

The sixth embodiment 6 can further comprise the features of the second embodiment 2.

The amount of time a user has been in fluid communication with the apparatus or breath time can be used to aid the calculation of the volume of a breath. For example, if the volume sensor 15 is a flow sensor, the flow sensor communicates fluctuations in a velocity of a breath to the processing means 12, whilst the timer 21 determines a breath time. The processing means 12 then determines an average velocity (AvVel) for the breath and uses the breath time in conjunction with the area of the fluid inlet 14 to calculate the volume of a breath (VOB):

VOB=(AvVel*area of fluid inlet)/breath time

The processing means 12 could comprise the timings means 21.

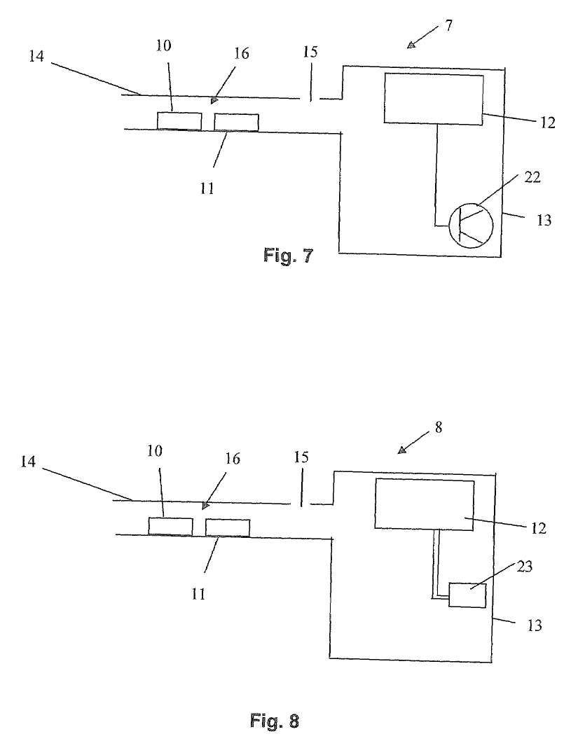

FIG. 7 shows the seventh embodiment 7 of the apparatus. The apparatus 7 comprises the features of the first embodiment and a warning means 22. The warning means 22 issues a signal when a predetermined number of breaths have been delivered to the apparatus 6.

The seventh embodiment 7 can further comprise the features of the second embodiment 2.

The warning means 22 comprises any suitable means or combination of means. For example, the warning means 22 could be an audible signal, a visual signal or a tactile signal.

FIG. 8 shows the eighth embodiment of the apparatus 8. The apparatus 8 comprises the features of the first embodiment and a breath counter 23. The breath counter 23 communicates a breath count to the processing means 12. The breath count is used by the processing means 12 to determine when a user has provided the correct number of breaths or the total number of breaths in a period of time. The processing means 12 then uses the value from the breath counter 23 to average the sensor readings and establish the type of fuel burnt by a user.

The eighth embodiment 8 can further comprise the features of the second embodiment 2.

For example, the processing means 12 can use the breath counter 23 to determine the average volume oxygen present in a user's breath. For example, if the breath counter 23 detects three breaths, the average volume of oxygen present in a user's breath (VO₂B) will be as follows:

VO₂B=(VO₂B1+VO₂B2+VO₂B3)/3

A similar calculation can be conducted for the carbon dioxide sensor.

There are a number of ways in which the apparatus 8 can provide a breath counter 23. On such way is to use the volume sensor 15 in conjunction with the processing means 12 as the breath counter 23. For example, when the volume sensor 15 senses a breath, the processing means 12 updates the breath counter 23.

Alternatively, the breath counter 23 can be a distinct sensor, such as a pressure sensor, and be self-updating.

In another alternative, the breath counter 23 could, for example, be provided using the oxygen sensor 10 and the carbon dioxide sensor 11 in conjunction with the processing means 12. In this alternative one way of providing the breath counter is to position the oxygen sensor 10 and carbon dioxide sensor 11 such that when a breath is exhaled or inhaled the processing means 12 is configured to detect the detection sequence, i.e., when a breath is exhaled into the apparatus 1 the oxygen sensor 10 detects the breath before the carbon dioxide sensor 11 and the processing means 12 is configured to determine this detection sequence to be a breath and updates the breath counter accordingly.

The first through eight embodiments 1-8 are merely exemplary and other embodiments are of course possible. Such embodiments can be made by combining any of the features of the first through seventh embodiments. For example, the sixth embodiment can be combined with the seventh embodiment and the warning means 22 configured to provide a signal to a user when the user has been in fluid communication with the device for a predetermined time.

Another possible combination is to combine the third and seventh embodiments. In such a combination where the device is configured for multiple users, the warning means can be used, for example, to warn a user when they have selected an incorrect user configuration.

FIG. 1 shows an apparatus 1 in which the housing 10 contains the processing means 12. However, other embodiments are of course possible where the processing means 12 is provided partially on a computer and in the housing 13. For example, the processing means 12 in the housing 13 could be provided to calculate the volume of oxygen present in a users breath and the processing means 12 on the computer is then used to adjust this value with, for example, the user parameters to calculate the user's metabolic rate. Alternatively, the processing means 12 could be provided on a computer and sensor readings are communicated directly to the computer.

Each apparatus 1-8 is handheld. However, the apparatus can also be desk mountable. The desk mountable apparatus comprises a flexible fluid inlet 13.

Each apparatus 1-8 can also comprise other sensors to improve its accuracy. The other sensors include humidity sensors, temperature sensors, altitude sensors, etc.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An apparatus for determining the type of fuel burnt by a user, the apparatus comprising an oxygen sensor and a carbon dioxide sensor, and wherein the oxygen sensor and the carbon dioxide sensor are operable to establish the type of fuel burnt by a user of the apparatus.

2. The apparatus according to claim 1, wherein the apparatus further comprises a volume sensor operable to establish the type of fuel burnt by a user.

3. The apparatus according to claim 1, wherein the apparatus further comprises a processing means configured to establish the type of fuel burnt by a user from at least one measurement from the oxygen sensor and at least one measurement from the carbon dioxide sensor.

4. The apparatus according to claim 3, wherein the processing means is configured to establish the type of fuel burnt by a user from at least one measurement from the volume sensor.

5. The apparatus according to claim 3, wherein the processing means is configured to determine the fuel burnt as a ratio of the carbon dioxide sensor measurement to the oxygen sensor measurement.

6. The apparatus according to claim 5, the ratio is a ratio of the amount of carbon dioxide produced by a user to the amount of oxygen consumed by a user.

7. The apparatus according to claim 5, wherein the ratio is a ratio of the volume of carbon dioxide produced by a user to the volume of oxygen consumed by a user.

8. The apparatus according to claim 5, wherein the processing means is configured to compare the ratio to predetermined respiratory quotient values to determine the type of fuel burnt by a user.

9. The apparatus according to claim 5, wherein the processing means is configured to match the ratio to a predetermined respiratory quotient value to establish the type of fuel burnt by a user.

10. The apparatus according to claim 1, wherein the apparatus further comprises a display configured to display type of fuel burnt by a user.

11. The apparatus according to claim 3, wherein the processing means is configured to establish the amount of oxygen consumed by a user from the amount of oxygen inhaled and exhaled by a user.

12. The apparatus according to claim 4, wherein the processing means is configured to establish the volume of oxygen consumed by a user from the volume of oxygen inhaled and exhaled by a user.

13. The apparatus according to claim 3, wherein the processing means is configured to establish the amount of carbon dioxide produced by a user from the amount of carbon dioxide inhaled and exhaled by a user.

14. The apparatus according to claim 4, wherein the processing means is configured to establish the volume of carbon dioxide produced by a user from the volume of oxygen inhaled and exhaled by a user.

15. The apparatus according to claim 3, wherein the processing means is configured to determine the oxygen consumed by a user by subtracting a inhaled breath measurement from a exhaled breath measurement.

16. The apparatus according to claim 3, wherein the processing means is configured to determine the carbon dioxide produced by a user by subtracting the exhaled breath measurement from the inhaled breath measurement.

17. The apparatus according to claim 3, wherein the processing means is configured to use a predetermined inhaled breath measurement to establish the oxygen consumed and carbon dioxide produced by a user.

18. The apparatus according to claim 1, wherein the apparatus comprises a timer configured to determine an inhaled breath time.

19. The apparatus according to claim 18, wherein the processing means is configured to determine the amount or volume of oxygen in an inhaled breath by multiplying the inhaled breath time by the appropriate predetermined inhaled breath measurement.

20. The apparatus according to claim 1, wherein the sensors are operable periodically.

21. The apparatus according to claim 1, wherein the sensors are operable continuously.

22. The apparatus according to claim 1, wherein the apparatus further comprises a breath direction sensor configured to establish if a user is inhaling or exhaling into the apparatus.

23. The apparatus according to claim 22, wherein the processing means is configured to use the breath direction sensor readings to classify the oxygen sensor and carbon dioxide sensor readings into exhaled or inhaled breath measurements.

24. The apparatus according to claim 22, wherein the processing means is configured to use the breath direction sensor readings to classify the volume sensor readings into exhaled or inhaled breath measurements.

25. The apparatus according to claim 1, wherein the apparatus is configured to determine the type of fuel burnt by a user from at least one breath.

26. The apparatus according to claim 1, wherein the apparatus is configured to determine the type of fuel burnt by a user from a plurality of breaths.

27. The apparatus according to claim 1, wherein the apparatus further comprises a timer, the timer configured to determine the amount time that a user is in fluid communication with the apparatus.

28. The apparatus according to claim 27, wherein the apparatus further comprises a warning means configured to warn the user when the have been in fluid communication with the device for a predetermined time

29. The apparatus according to claim 1, wherein the apparatus is configured to determine the amount or volume of carbon dioxide produced by averaging the carbon dioxide produced from each of the plurality of breaths.

30. The apparatus according to claim 29, wherein the amount or volume of carbon dioxide produced is determined by averaging the total amount or volume of carbon dioxide produced by a user with the total time that a user is in fluid communication with the apparatus.

31. The apparatus according to claim 1, wherein the total amount or volume of carbon dioxide produced is determined by averaging the total amount or volume of carbon dioxide produced with the total number of breaths a user delivers to the apparatus.

32. The apparatus according to claim 1, wherein the apparatus further comprises a housing and a first and a second fluid inlet.

33. The apparatus according to claim 32, wherein the first fluid inlet is arranged in fluid communication with the oxygen sensor and/or the carbon dioxide sensor.

34. The apparatus according to claim 32, wherein the second fluid inlet is arranged in fluid communication with the oxygen sensor and/or the carbon dioxide sensor.

35. The apparatus according to claim 32, wherein the first fluid and/or the second fluid inlet is arranged in fluid communication with the breath direction sensor.

36. The apparatus according to claim 32, wherein the oxygen sensor and/or the carbon dioxide sensor are arranged between the first fluid inlet and the second fluid inlet.

37. The apparatus according to claim 32, wherein the breath direction sensor is arranged between the first and the second fluid inlets.

38. The apparatus according to claim 32, wherein the first fluid inlet comprises the second fluid inlet.

39. The apparatus according to claim 32, wherein the first fluid inlet is a tube.

40. The apparatus according to claim 39, wherein the tube is rigid.

41. The apparatus according to claim 39, wherein the tube is flexible.

42. The apparatus according to claim 39, wherein the second fluid inlet is provided as at least one aperture on the tube.

43. The apparatus according to claim 1, wherein the apparatus further comprises a housing.

44. The apparatus according to claim 43, wherein the housing comprises the first fluid inlet.

45. The apparatus according to claim 43, wherein the housing comprises the second fluid inlet.

46. The apparatus according to claim 43, wherein the first and second fluid inlets are arranged in fluid communication with the oxygen sensor and/or the carbon dioxide sensor.

47. The apparatus according to claim 43, wherein the first and second fluid inlets are arranged in fluid communication with the breath direction sensor.

48. A method of calculating the type of fuel burnt by a user, the method comprising the steps of:
providing a apparatus comprising an oxygen sensor and a carbon dioxide sensor;
establishing the proportion of oxygen consumed by the user;
establishing the proportion of carbon dioxide produced by a user; and
establishing the type of fuel burnt by a user.

49. The method according to claim 48, wherein in step (a) the apparatus provided is the apparatus according to the first aspect and the proportions established in steps (b) and (c) are an amount of oxygen consumed and carbon dioxide produced by a user.
